(19) United States
(12) Patent Application Publication
(10) Pub. No.: US 2024/0249805 A1
(43) Pub. Date: Jul. 25, 2024

(54) SYSTEM AND METHOD FOR CREATING A DIGITAL VIRTUAL SPONSOR

(71) Applicant: Addiction Resource Systems, Inc., Aventura, FL (US)

(72) Inventors: Hayes THOMAS, Aventura, FL (US); Matthew Charles REEDY, San Antonio, TX (US)

(73) Assignee: Addiction Resource Systems, Inc., Aventura, FL (US)

(21) Appl. No.: 18/427,613

(22) Filed: Jan. 30, 2024

Related U.S. Application Data
(63) Continuation-in-part of application No. 16/441,886, filed on Jun. 14, 2019, now abandoned.
(60) Provisional application No. 62/685,047, filed on Jun. 14, 2018.

(51) Int. Cl. G16H 10/60; G06N 20/00

(52) U.S. Cl. CPC G16H 10/60; G06N 20/00

(57) ABSTRACT

Methods and systems including computer programs encoded on computer storage media, for creating a digital virtual sponsor are disclosed. One of the methods includes receiving an inquiry from a person struggling with addiction. The inquiry is analyzed to determine a response category. At least one appropriate response to the inquiry is determined based on a scripted interaction associated with the response category and a response machine learning model. The at least one response to the inquiry is provided to the person struggling with addiction.

### CROSS-REFERENCE TO RELATED APPLICATIONS

This application is a Continuation-in-Part of copending application Ser. No. 16/441,886, filed on Jun. 14, 2019, which claims priority under 35 U.S.C. § 119(c) to U.S. Provisional Application No. 62/685,047, filed on Jun. 14, 2018, all of which are hereby expressly incorporated by reference into the present application.

### BACKGROUND OF THE INVENTION

This specification relates to using natural language processing and machine learning to help facilitate individual addiction recovery.

Recovery programs are designed to help individuals recover from addiction. These programs provide individuals with accountability and incentives for taking necessary steps and actions to address their addictions. For example, a twelve-step program includes a set of guiding principles outlining steps for recovery including: admitting that a person cannot control addiction; recognizing a higher power that can give strength; examining past errors with the help of a sponsor; making amends for these errors; learning to live a new life with a new behavior code; and helping others who suffer from the same addictions.

Staying on a recovery program can be extremely difficult for a person struggling with addiction. In order to maintain sobriety, a person needs to stay motivated. Often, individuals with addictions rely on others to help them with this motivation, meeting regularly with a sponsor or group of people and working through the recovery steps together.

Sometimes individuals struggling with addiction cannot feasibly meet in person with a sponsor or attend group meetings, or need more support than is provided in groups or with sponsors. These individuals need daily accountability and incentives to help them stay motivated in the recovery process.

The current methods/systems and technology of addiction recovery solutions focus primarily on education in a classroom setting based on a Matrix curriculum that overloads the person struggling with addiction with information and minimal to no guidance, motivation and/or updates on the status of the person's progress throughout the recovery process. Further, current methods and systems for addiction recovery merely provide the person with directions to recovery meetings and/or connect the person to a counselor. These described services are not available 24/7 nor can they provide conversational support in “real-time”, to help the person at the most critical times in addiction recovery.

These deficiencies in the current methods, systems and technology for addiction recovery result in an ineffective addiction recovery model, as evidenced by an approximately 2% success rate for persons in rehabilitation programs for addiction. Specifically, this mere 2% rate of success among persons in rehabilitation programs is attributed to the lack of “real-time” engagement, motivation and/or support in existing addiction treatment methods and systems.

As such, there is a need for a system and method for addiction recovery that solves the aforementioned deficiencies in the state of the art.

### SUMMARY OF THE INVENTION

This specification describes technologies for using natural language processing and machine learning to help facilitate individual addiction recovery. These technologies generally involve a digital virtual sponsor system and method for creating a digital virtual sponsor to hold a person struggling with addiction accountable for his or her actions and to provide incentives to encourage the person to make good choices on the steps to recovery without requiring the person to attend group meetings or meet with a sponsor in person.

As will be apparent in the disclosure that follows, embodiments of the invention are generally directed to a method and system that employ an algorithm and Artificial Intelligence (AI) platform that use a series of machine learning models that are continually trained (i.e., trained and retrained), based on prior interactions with a user/person, information in a knowledge database and/or other resources. The described AI platform and continually-trained machine learning models provide an improvement to the computer technology and existing technologies for addiction recovery, since the AI platform and trained machine learning models adapt with immediacy to user response, thus being more efficient and user/person focused, to provide responses in a recovery plan tailored to the specific user/person in “real-time”, based on the specific status of addiction, state-of-mind and/or intent of the user/person at a specific moment.

These described embodiments of the invention allow for a significant improvement in the success rate of persons enrolled in addiction recovery programs, as compared to existing methods, systems and technologies for addiction recovery. Further, the described method and system achieve additional advantages by providing “real-time” responses, feedback and/or status reports regarding the progress of the person in an addiction recovery program, to any third-parties including, but not limited to, agencies, courts, schools and treatment facilities.

In an embodiment of the invention, a digital virtual sponsor system includes a digital assistant platform that interacts with an individual using natural language processing to create a dialogue with the individual regarding sobriety choices and recovery behavior, a knowledge base that contains information and resources about addiction and recovery, and machine learning models that use blockchain data and the knowledge base to provide the individual with personalized answers, resources, sobriety challenges, and other necessary information to facilitate recovery.

In general, one innovative aspect of the subject matter described in this specification can be embodied in systems and methods to provide a digital virtual sponsor for people struggling with addiction.

The foregoing and other embodiments can each optionally include one or more of the following features, alone or in combination. In particular, one embodiment includes all the following features in combination.

An example system includes a digital assistant platform that allows communication with a person struggling with addiction using natural language conversations; a knowledge base containing information about resources, activities, and recovery plans to help the person struggling with addiction; and a machine learning subsystem configured to: receive a user inquiry from the digital assistant platform, analyze the inquiry to determine a response category using a natural language machine learning model, determine at least one appropriate response to the inquiry based on a scripted interaction associated with the response category and a machine learning model trained for recovery plan responses using the knowledge base, and provide the at least one response to the inquiry to a user using the digital assistant platform.

An example method includes receiving an inquiry from a person struggling with addiction; analyzing the inquiry to determine a response category using a natural language machine learning model; determining at least one appropriate response to the inquiry based on a scripted interaction associated with the response category and a machine learning model trained for recovery plan responses; and providing the at least one response to the inquiry to the person struggling with addiction.

The inquiry may be a request for information, a statement for motivation, or a warning sign that the person needs help to continue a recovery process. The inquiry may be a spoken request and analyzing the inquiry using natural language processing to determine the response category.

The scripted interaction may be a set of interactive dialogue prompts that is created using a predefined recovery plan and a profile associated with the person. The scripted interaction may be updated using information from the machine learning model based on responses from previous interactive dialogue with the person and updates to the person's profile.

Another example method may include: providing a sobriety challenge to the person struggling with addiction using a networked device; receiving a response to the sobriety challenge from the networked device; determining whether the person struggling with addiction successfully completed the sobriety challenge using information received from the networked device; determining sobriety of the person struggling with addiction based on a completion state of the sobriety challenge; and providing a response based on the sobriety state of the person struggling with addiction using the networked device.

Determining whether the person struggling with addiction successfully completed the sobriety challenge includes determining that the person successfully completed the sobriety challenge. The person may be provided with a digital award after successfully completing the sobriety challenge. A second sobriety challenge may be provided at a later time during the same day to ensure the person is maintaining sobriety.

Determining whether the person struggling with addiction successfully completed the sobriety challenge includes determining that the person unsuccessfully completed the sobriety challenge. In this case, a dialogue may be initiated with the person struggling with addiction to encourage the person to follow a recovery plan. An alert may be sent to at least one contact of the person struggling with addiction. At least one credit card of the person struggling with addiction may be canceled so that the person is not tempted to give in to the addiction.

Other embodiments of this aspect include corresponding computer systems, apparatus, and computer programs recorded on one or more computer storage devices, each configured to perform the actions of the methods. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

The subject matter described in this specification can be implemented in particular embodiments so as to realize one or more of the following advantages.

A digital virtual sponsor system provides accountability and incentives for a person struggling with addiction without the person needing to be physically present in group meetings or with a sponsor. The system uses natural language and machine learning to provide sobriety challenges, resources, and accountability to the recovering person. The system can also provide incentives for reaching certain milestones, getting through a day without relapsing, following recovery steps, and encouragement to stay sober. The system can further reach out to the struggling person if the system determines that the person is not following his or her recovery program or at risk of relapse. The system can suggest recovery activities and information and also connect with the person's contacts in order to ensure the person's safety and sobriety.

In addition to incentives, the system enables friends of the recovering person to pose recovery challenges to the recovering person. A leaderboard can also indicate the progress the recovering person is making as compared to his or her friends.

The system can also provide information to third parties, e.g., courts or sponsors regarding the recovering person's successes and failures. Additionally, the system may collect high quality data for research and analytics to find root causes for relapse. The system can further provide a judgment-free resource so that the recovering person can feel comfortable reaching out to the system for information and help in the recovery process.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 illustrates a digital virtual sponsor system that uses natural language processing and machine learning to facilitate virtual sponsorship with incentives and accountability.

FIG. 2 illustrates an example dialogue between a person struggling with addiction and a communication device of the digital virtual sponsor system.

FIG. 3 illustrates examples of two dialogues between a person and the system that have been tailored to the person based on user profile information and previous interactions.

FIG. 4 is a flowchart of an example process for providing a digital virtual sponsor to facilitate addiction recovery.

FIG. 5 is a flowchart of an example process for determining sobriety of a person struggling with addiction.

FIG. 6 is a flowchart of an example process performed by machine learning models 150 to determine a response to an inquiry received from person 101.

FIG. 7 illustrates exemplary steps performed by machine learning model 150 to analyze inquiries from person 101 and determine appropriate responses.

Like reference numbers and designations in the various drawings indicate like elements.

### DETAILED DESCRIPTION OF CERTAIN EMBODIMENTS

The specification generally describes a digital virtual sponsor system that provides dynamic, interactive dialogue, sobriety challenges, and incentives to a person struggling with addiction.

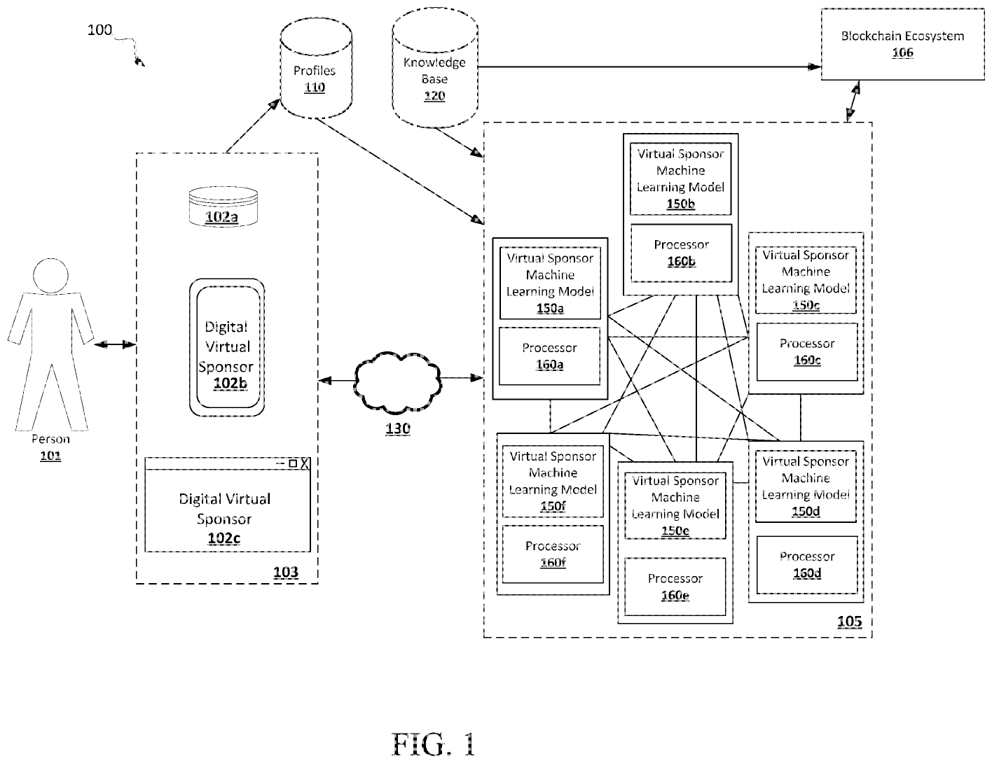

FIG. 1 illustrates an example digital virtual sponsor system 100 that engages in dynamic, interactive dialogue with a person struggling with addiction to support the person's recovery, provide accountability, and incentivize the person to make good choices. The digital virtual sponsor system 100 is an example of a system implemented as computer programs on one or more computers in one or more locations, in which the systems, components, and techniques described below can be implemented.

In some implementations, a person struggling with addiction 101 registers with the digital virtual sponsor system 100, e.g., using a web browser navigated to a web page of the digital virtual sponsor system 100 or communicates in a batch form or iteratively over time with a digital assistant to complete a profile. The person 101 can access the web page using a computing device 102c. During registration, the person 101 provides basic information to create a user profile within the system 100. The information provided by the person 101 may include: name, age, family conditions, address, contact information, emergency contact information, faith affiliation, addiction struggle, and sobriety time. Other information can be added to the user's profile, either at the time of initial creation or as the user interacts with the system 100. This information includes: information pertaining to addiction that is categorized by a recovery program and information provided by the user's victims, family, friends, and community.

Once the person 101 is registered with the system 100, the person 101 can use one or more connected devices including Internet of Everything (IoE) devices using a communication platform 103 to access the system 100 in order to obtain support for his or her recovery process. In some implementations, the person 101 accesses the digital virtual sponsor system 100 using e.g., a smartphone 102b, a computing device 102c, or a smart home device, e.g., a digital assistant, 102a. Although FIG. 1 illustrates a number of devices, other devices such as tablets, smart devices, a desktop or laptop computer, a mobile device, a wearable device e.g., a virtual reality headset, home and building automation devices, a gas station pump equipped with digital assistant technology support, or other networked devices can facilitate interaction between the person 101 and the digital virtual sponsor system 100.

A person 101 can interact with the digital virtual sponsor system 100 by e.g., typing, texting, using a touch screen, or conversing with the system 100 using natural language.

In one implementation, the person 101 can query the system 100 using natural, conversational language. The communication platform 103 uses a digital assistant, e.g., Amazon Alexa, Siri® from Apple, Google Home®, Microsoft Cortana®, or any one of various others, to process the natural language and understand the person's inquiry. In some instances, the query can be in the form of a question, e.g., “Alexa, what time is the recovery meeting tonight?” In other instances, the query can be a statement, e.g., “Hey Google, I think I need a drink.”

Regardless of the form of the inquiry, the system 100 receives the query from the person 101 using the communication platform 103 and processes the query using a machine learning subsystem 105 that includes virtual machine learning response models 150a-150f and processors 160a-160f for determining appropriate responses. The machine learning models are trained on example conversations with users to determine the intent of the users and the proper category for response. For example, the question “Alexa, what time is the recovery meeting tonight?” may be categorized as an information query whereas the statement “Hey Google, I think I need a drink” may be categorized as a motivation or warning signs inquiry, depending on follow-on conversation with the user.

The models then predict the proper response to user queries given a predefined set of steps for different response categories. Each response category can have an initial defined template that includes the response steps. These steps can evolve and adapt to each person individually the system 100 gains more information regarding people over time. For example, one recovering person may respond better to mediations while another recovering person may respond better to breathing exercises. The system learns behavior of each user and tailors responses to each user's individual preferences and responses.

In some implementations, the machine learning subsystem 105 includes different types of machine learning models, e.g., one for natural language processing, a second type for determining an appropriate response to received natural language, and/or one for predicting whether a user will relapse. For example, the machine learning subsystem 105 may be WingMan as described in U.S. Patent Application No. 62/827,615, for DIGITAL VIRTUAL SPONSOR, which was filed on Apr. 1, 2019, and which is incorporated here by reference.

In some implementations, the machine learning models 150a-150f are neural networks. Neural networks are machine learning models that employ one or more layers of neurons to generate an output, e.g., one or more classifications, for a received input. Neural networks may include one or more hidden layers in addition to an output layer. The output of each hidden layer can be used as input to the next layer of the network, i.e., the next hidden layer or the output layer, and connections can also bypass layers, or return within the same layer such as in the case of a recurrent network. Each layer of the neural network generates an output from its inputs in accordance with the network architecture and a respective set of parameters for the layer.

In addition to typical weights and biases, networks may include gates to hold memory as well as gates to remove data from memory such as in a Long Short-Term Memory (LSTM) network. A stateful network such as the LSTM aids in sequence classification and allows the network to understand the context of current data based on prior events.

The machine learning subsystem 105 may use machine learning libraries to develop, train and retrain the machine learning models 150a-150f, as needed in accordance with the described embodiments of the invention. For example, the system 100 may incorporate Rasa NLU and tensorflow to determine user intents and how user messages or queries should be categorized for natural language processing machine learning models and to develop and train response and predictive models.

The machine learning subsystem 105 uses information from a knowledge base 120 and user profiles 110 to train and/or retrain response and relapse-predictive models to provide responsive dialogue for user questions and issues related to overcoming addiction issues. The knowledge base 120 is created through (1) public information on addiction and resources, e.g., currently available information from books, websites, recovery program material etc.; (2) private information on addiction and resources, e.g., information from sponsors, group members, family members, and other addicts; and (3) machine-generated information from computer applications. Information includes advice, recommendations, guidance, and interactions that have been proven helpful previously to recovering persons and that may be valuable in a current context, e.g., a conversation with a person struggling with addiction. It shall be appreciated that machine learning subsystem 105 is not limited to accessing information from the knowledge base 120, and can therefore access information from any number of additional knowledge bases and/or data storage devices, as needed to implement the functionality of the described embodiments of the invention.

The system 100 additionally includes a blockchain ecosystem 106 that collects and tracks the progresses of users and information in the recovery process. In some implementations, the blockchain ecosystem 106 separates good information from bad information. The information may be separated using e.g., predefined criteria, expected results vs. actual results, or other data points. In other implementations, instead of a blockchain ecosystem any database may be used. The information can be uploaded to the machine learning subsystem 105 and used in further processing. In some implementations, this information can be used in the training and execution of the machine learning models 150a-150f in the machine learning subsystem 105. In an implementation, only good information is uploaded to the machine learning subsystem 105. In another implementation, both good and bad information is uploaded to the machine learning subsystem 105. The data may be labeled as good or bad so that it can be used appropriately, e.g., good information may be used to increase the values of certain machine learning parameters while bad information may be used to decrease the values of certain machine learning parameters.

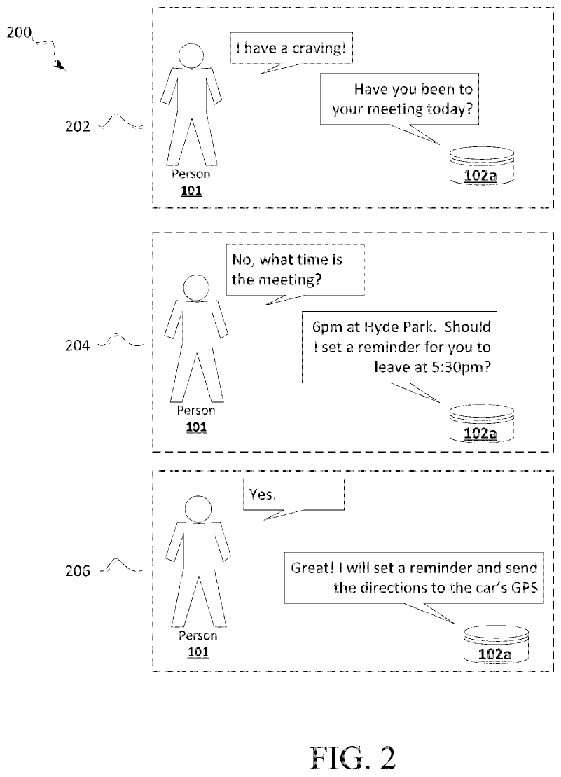

FIG. 2 illustrates an example dialogue between a person struggling with addiction and a communication device 102a of the digital virtual sponsor system 100. The dialogue 200 is broken into three interactions 202-206 of back and forth dialogue between the person 101 and the communication device 102a. In a first interaction 202, a person 101 states that he has a craving. The system 100 classifies this inquiry or received statement from the person using the natural language machine learning models 150a-150f illustrated in FIG. 1.

In this example, the system 100 categorizes the inquiry as a warning sign. The system 100 then provides scripted information regarding dialogue for warning sign behavior from the person 101. The scripted interaction may be a set of interactive dialogue prompts that is created using a predefined recovery plan and a profile associated with the user. In some implementations, the scripted information has been adapted for the particular person 101 based on profile information, information from the knowledge base 120 and/or other information obtained from previous interactions with the system 100. The system 100 uses additional machine learning models to determine the appropriate response to the person's initial statement and predict whether a person will relapse. The system 100 can update the scripted interactive dialogue using information from the machine learning models and/or updates to users' profiles.

In this example, after categorizing the person's statement as a warning sign, the system 100 determines that the person 101 needs accountability in order to maintain sobriety. Therefore, the system 100 asks the person 101 whether the person has attended a sobriety meeting today.

In a second interaction 204, the person 101 answers the system's 100 query regarding whether he has attended his sobriety meeting. The person 101 then follows up with a question regarding the time of the meeting. The system 100 then answers the question and offers to set a reminder in order for the person 101 to leave on time.

In a final interaction 206, the system 100 responds to the person's positive response to set a timer and sends directions to the meeting to the person's car.

The system 100 can know that the person 101 is in the car on the way to the meeting, has arrived at the meeting, or is leaving the meeting using any of the person's 101 networked-devices, e.g., devices that are connected using the Internet, Bluetooth, NFC, cellular network, or other network. These devices can include wearables, smartphones, and wireless-enabled sensors in the car. Once the system 100 determines that the person 101 followed the recovery plan and attended the meeting, the system can reward the person, e.g., using digital currency such as Noiacoin. The Noiacoin (NCN), like any accepted cryptocurrency can be traded on any supported exchange for any supported crypto trading pair, e.g., NCN/BTC, NCN/ETH, and eventually for fiat. Award/and or credits may be given towards time served, or requirements handed out by courts, therapists, rehabilitation centers, etc.

Additional rewards include badges and progression up a leaderboard with other users of the system 100, e.g., friends of the person struggling with addiction.

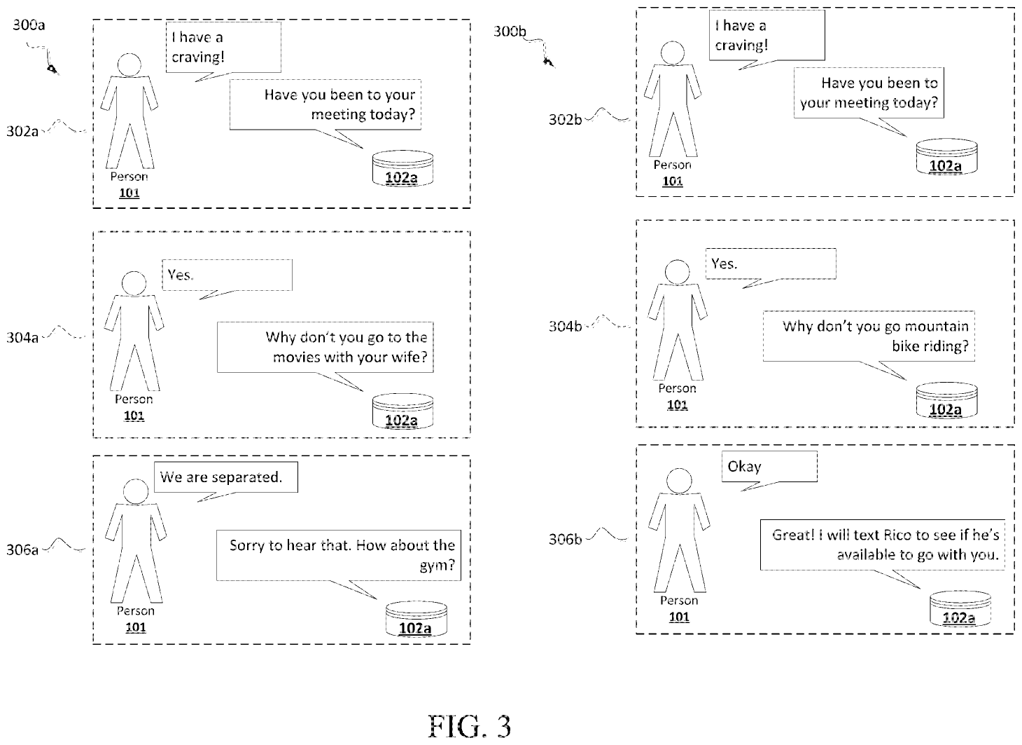

FIG. 3 illustrates examples of two dialogues between a person 101 and the system 100 that have been tailored to the person based on user profile information and previous interactions. Dialogue 300a represents a conversation that occurs with the person at a date prior to the conversation occurring in Dialogue 300b. In Dialogue 300a, after the person 101 has stated that he has in fact attended his meeting for the week, and yet is still having cravings, the system 100 suggests that he do something with his wife 304a. However, in the next interaction 306a, the system 100 learns from the person that he and his wife are separated. The system 100 then makes an alternative suggestion.

Dialogue 300b illustrates that the system 100 has learned from the previous interaction in Dialogue 300a so that when the person 101 states that he has a craving and has already attended his meeting, the system 100 no longer suggests activities with the person's wife. Instead, the system 100 can pull information from the person's profile that is saved in a database in the system 100, containing answers to the initial registration questions as well as pertinent information that the system 100 has learned about the person 101 over time. In this example, the system 100 tailors the response to the person 101 by knowing that the person 101 likes to mountain bike ride and suggests this activity. The system 100 can also have access to the person's contact information from other networked devices and then suggest a friend to join the person 101 on the ride.

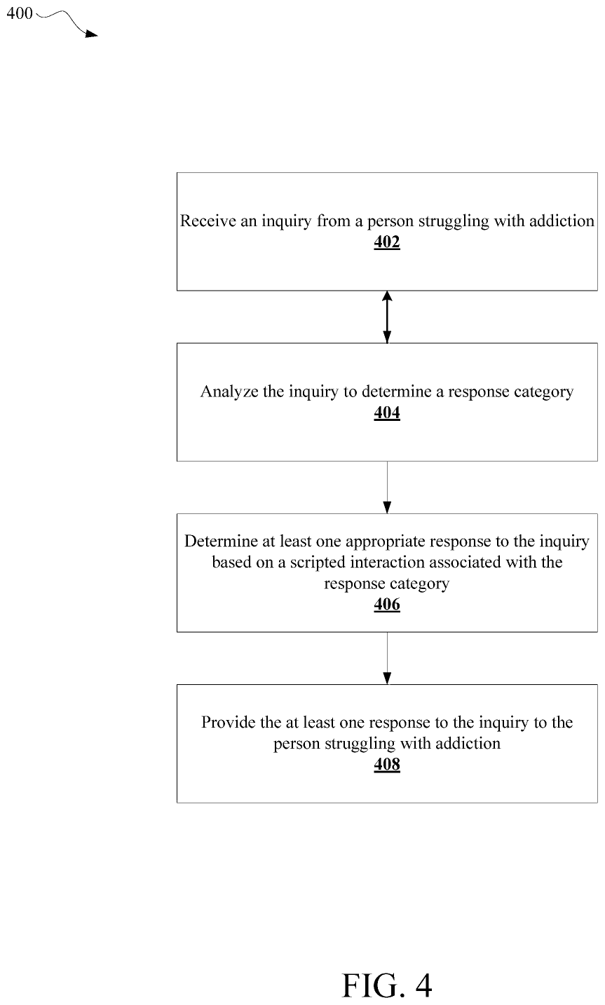

FIG. 4 is a flowchart of an example process 400 for providing a digital virtual sponsor to facilitate addiction recovery. For convenience, the process 400 will be described as being performed by a system of one or more computers, located in one or more locations, and programmed appropriately in accordance with this specification. For example, a digital virtual sponsor system, e.g., the digital virtual sponsor system 100 of FIG. 1, appropriately programmed, can perform the process 400.

First, the system 100 receives an inquiry from a person struggling with addiction 402. The system analyzes the inquiry to determine a response category 404. As described above, the response category can be any category determined by natural language machine learning models e.g., information, motivation, or warning-sign. The system then determines at least one appropriate response to the inquiry based on a scripted interaction associated with the response category 406. The system 100 uses machine learning, user profiles, the knowledge base, and previous interactions with the user (i.e., both inquiries received from the user and tailored responses to the inquiries as determined by the system 100) to determine the appropriate response to the present given inquiry. The machine learning and knowledge base elements of the system 100 aggregate the actual experiences of a large number of recovering persons and their responses to determine specific recommendations and responses. The user profile and previous interactions of the user with the system provide personalized information about the user to the machine learning subsystem 105, which uses the personalized information to identify responses that are most effective for individuals having the same attributes as that particular user.

The system then provides the at least one response to the inquiry to the person struggling with addiction. The response can be provided using the communication platform that the person used to communicate with the system or any number of other networked devices that are programmed to accept a response.

The response may include suggestions or activities for avoiding addictive behavior. The system can incentivize positive behavior that encourages recovery by providing rewards for following the suggestions, participating in the activities, or otherwise accomplishing recovery goals. For example, if the system 100 determines that the person struggling with addiction has taken the suggestions or done the recommended activities, the system can reward the person with digital currency or other incentives. The digital currency may be provided in different amounts depending on the amount the system 100 allocates for each suggestion, activity, or personal accomplishment.

In some implementations, the system 100 can initiate communication with a person struggling with addiction to ensure that the person is maintaining sobriety.

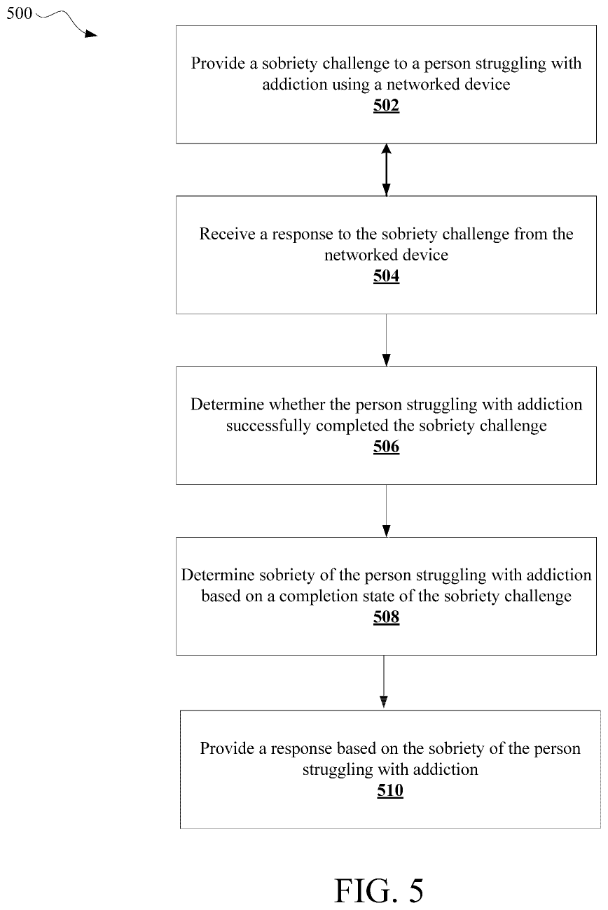

FIG. 5 is a flowchart of an example process 500 for determining sobriety of a person struggling with addiction. For convenience, the process 500 will be described as being performed by a system of one or more computers, located in one or more locations, and programmed appropriately in accordance with this specification. For example, a digital virtual sponsor system, e.g., the digital virtual sponsor system 100 of FIG. 1, appropriately programmed, can perform the process 500.

The system 100 provides a sobriety challenge to the person struggling with addiction using a networked device 502. The sobriety challenge can be a question that the person needs to answer, e.g., “Have you attended your meeting?” or “Have you talked with your sponsor?” The sobriety challenge can also be a puzzle, e.g., a brain teaser, Sudoku, or other mentally challenging activity that can be issued digitally and returned to the system. Any challenge may be used that allows the system to determine if a user is clean and sober or if the user has started using again. The sobriety challenge can be issued on regular intervals such as daily, hourly, twice a day, or some other predefined time period in order to ensure sobriety of an individual.

The system 100 then receives a response to the sobriety challenge 504. In some implementations, the sobriety challenge will have a time limit for response. Either the person to whom the sobriety challenge was sent will return the challenge to the system in the time limit or the system will register a timeout response. The system 100 determines whether the person successfully completed the sobriety challenge 506. The system 100 can do this by comparing the sobriety challenge answer with a correct answer if the challenge was a puzzle, checking with the meeting logs or sponsor to determine if the person went to a meeting or met with the sponsor, or using other records to verify the response of the person. The system can obtain information to verify the sobriety challenge answer using networked devices, e.g., IoE devices belonging to the person or the person's contacts.

After determining whether the person struggling with addiction successfully completed the sobriety challenge, the system determines the sobriety of the person based on the completion state of the sobriety challenge 508. The system can then provide a response based on the sobriety of the person struggling with addiction.

If the person successfully completed the sobriety challenge, the system determines that the person is sober. The system may then reward the person with a digital award, e.g., digital currency or some other incentive. The system may also schedule a second sobriety challenge for a time period in the future.

If the person does not successfully complete the challenge or the challenge times out before the person responds, the system determines that the person is not sober. People struggling with addiction often cheat for a few days before completely relapsing. The system can use a threshold amount of time of failed challenges or unresponsiveness to determine that a user is in danger of relapsing or has relapsed. The system may then try to engage the person in dialogue to determine how to help the person to follow recovery steps. In some implementations, the system determines the events that occurred in the person's life prior to relapsing in order to predict relapses in the future, and to help avoid such relapses again through new suggestions, therapy, information, or other recovery techniques.

The system may also try to contact people from the person's contact list in order to have the contacts encourage the person to follow the recovery steps. The system may also take measures to stop the person from spiraling into addiction, e.g., canceling credit cards, disabling vehicles, locking cabinets, and freezing bank accounts, notifying appropriate persons, e.g., family, friends, officials, etc. In some implementations, the system requires opt-in permissions from the user or mandated by a third-party and agreed to by the user prior to taking such measures, collecting user data, and/or notifying others of the user's progress.

The system can provide resources and information regarding how to help the person in his or her current state if they have slipped back into addiction. For example, if the person is homeless or jobless, the system can suggest temporary housing or jobs in the person's skillset. The system can provide any number of resources and accountability to support the person's recovery.

In addition to system-issued challenges, the system 100 can allow friends or contacts of the person struggling with addiction to pose recovery challenges and activities in which the participants compete against each other. A leaderboard can indicate the progress of all participants as compared to one another and show winners of challenges.

With consent of registered users, the system can additionally collect data for research and analytical analysis. This data can be used to determine a root cause for relapse among individual recovering persons.

As shall be appreciated in the described embodiments, system 100 is advantageous because it provides more tailored and relevant responses to a person 101 (or user) over time. For example in the system 100, a specific first response to a first inquiry from the person 101 is determined using one or more machine learning models 150a-150f, the knowledge base 120 is updated with information associated with the determined specific first response to the first inquiry, and a specific second response to a second inquiry from the person 101 is determined using the one or more machine learning models 150a-150f, which are retrained based on the specific prior-determined first response and associated information that are added as an update to the knowledge base 120.

As a result, the one or more machine learning models 150a-150f are continually trained and retrained, to determine specific recovery plan responses to inquiries from the person 101 that are unique to the person struggling with addiction, based on a plurality of previous interactions with the person (i.e., information on prior determined responses to the person's inquiries) and the knowledge base 120 comprising information on actual experiences of a plurality of other persons with successful recovery from addiction.

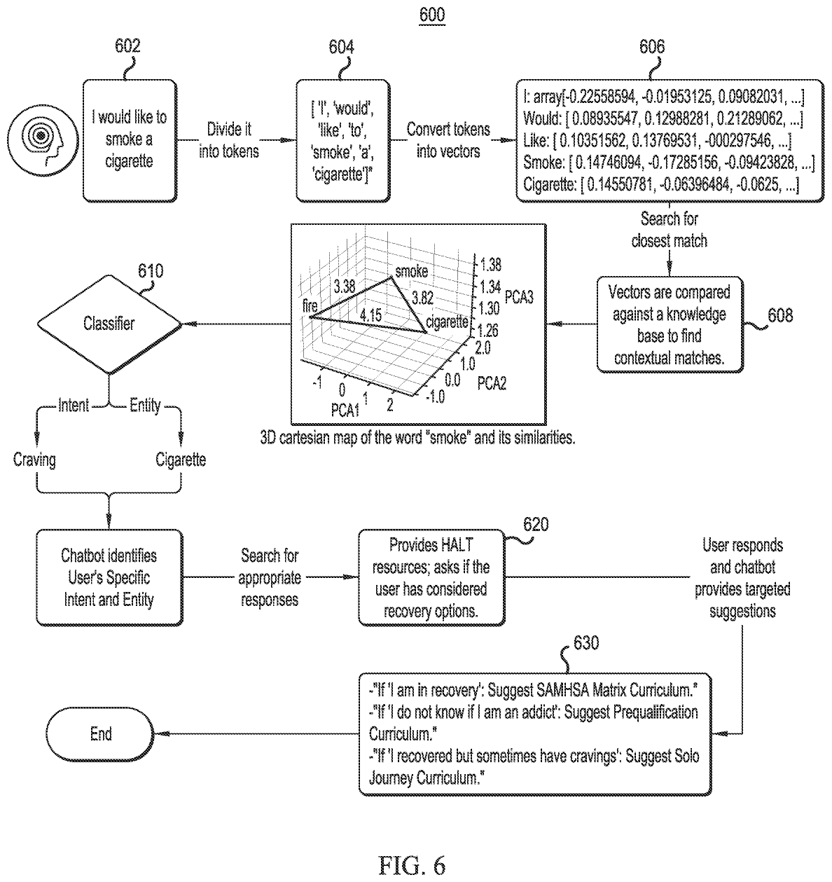

In one embodiment of the invention, FIG. 6 is a flowchart of an example process 600 performed by one or more (trained or retrained) machine learning models 150 to determine a response to an inquiry received from person 101 via the system 100. For simplicity, one or more machine learning models 150 is referred to as machine learning model 150 in the singular form.

At step 602 of process 600, machine learning model 150 receives an input phrase corresponding to an inquiry from a person 101. For example, the input phrase is a statement such as, “I would like to smoke a cigarette.” At step 604, machine learning model 150 performs a tokenization of the input, in which the inputted statement is divided into individual components (i.e., tokens) to perform an analysis. Therefore the statement, “I would like to smoke a cigarette.”, is divided into corresponding tokens so that each token corresponds to a single word (i.e., “I”, “would”, “like”, “to”, “smoke”, “a”, “cigarette”). At step 606, each of the tokens associated with one of the words in the statement is converted into a plurality of vectors.

This allows machine learning model 150 to perform a semantic analysis of the tokens, so that the system 100 can understand the meaning of each word in the context of the entire inputted statement. More specifically at step 608, machine learning model 150 compares the vectors for each of the corresponding tokens with contents of the most updated knowledge base 120, or any other additional knowledge base and/or data storage devices storing relevant information to perform the semantic analysis. This comparison of analyzed tokens with the corresponding knowledge base enables a determination of relevant contextual matches, which is critical in understanding nuances of the inquiry/statement of the person 101.

In one embodiment of the invention, each of the numeric values of the vectors for each token is searched in the knowledge base for a corresponding matching numeric value. The corresponding numbers in the knowledge base that are closest to the input numeric values of the vectors for each token are selected. These selected numbers are connected to the meanings of words, and in particular, matching words that pertain to addiction, addiction recovery, interpersonal relationships and psychology. This collection of matching words provides context for the person's inputted statement, and identifies a specific intent such as a craving, expression of a sentiment, and/or question about addiction recovery and rehabs.

At step 610, machine learning model 150 performs the described semantic analysis to perform a classification analysis that identifies the intent and any entities of the inputted statement/inquiry from the person 101. In the described example, the model determines that the statement, “I would like to smoke a cigarette.”, has a specific intent and expression of a craving to smoke. Further, machine learning model 105 recognizes “cigarette” as a key entity of the context of the statement. As a result, the model determines that the person 101 is expressing a desire to smoke a cigarette based on the inputted inquiry/statement.

At step 620, machine learning model 150 searches for an appropriate response to the inputted statement/inquiry from the person 101 based on the determined intent of the inquiry, the updated knowledge base 120, any alternative knowledge base and/or data storage device. In one embodiment, the determined intent of the inquiry is used to search for addiction specific responses in the knowledge base, which correspond to responses that have been created by experts in addiction recovery (that provide specific and relevant information to the person 101).

Based on the described example, machine learning model 150 can determine the response to the inputted statement from the person 101 to be any statement or offer of resources related to the HALT (Hungry, Angry, Lonely, Tired) strategy, which is beneficial in addiction recovery. Further, machine learning model 150 and machine learning subsystem 105 can determine an appropriate response that inquires if the person 101 has considered recovery options, such as a message of, “It's important to recognize what's driving your craving. Have you considered recovery options?”

At step 630, machine learning model 150 can determine additional tailored responses based on follow-up responses and/or inquiries from the person 101. For example, if the person 101 provides a statement, inquiry or indication of being in recovery, machine learning model 150 determines a subsequent response to the person 101 that suggests the SAMHSA Matrix Curriculum, which is designed for individuals actively engaged in recovery. Further, if the person 101 inputs a statement, inquiry or indication of uncertainty as to whether he/she is an addict, machine learning model 150 determines a subsequent response to the person 101 that suggests a prequalification curriculum to help assess the individual's relationship with certain substances. If the person 101 inputs a statement, inquiry or indication of being recovered but acknowledges the presence of occasional cravings, machine learning model 150 determines a subsequent response to the person 101 that suggests a Solo Journey Curriculum, which is aimed at supporting individuals in maintaining their recovery.

In the described embodiments of the invention, system 100 and the corresponding machine learning models learn more about the person 101 with each interaction, and tailor each subsequent response based on information that has been learned. As a result, each determined response is provided as an output to the person 101 that most closely applies to the individual's recovery plan, state of mind, progress through recovery and previous inputs.

For example, if the person 101 inputs a statement that he/she has a problem with triggers, the system will provide subsequent responses that provide guidance on how to manage the triggers. If the person 101 inputs a statement or provides an indication that he/she does not wish to consider Alcoholics Anonymous (AA) as a recovery aid, the system will not offer AA as a suggestion in subsequent responses.

In one embodiment of the invention, responses determined by machine learning model 150 are categorized into various recovery plans, which have been developed through research and experience by experts in addiction recovery. Notably, the recovery plans are created based on other addict's experiences and the steps each person took to achieve a successful recovery.

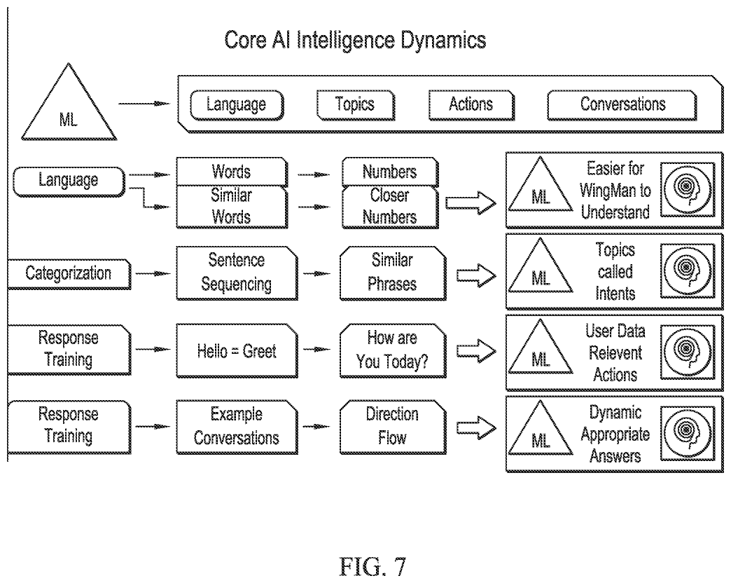

FIG. 7 provides an overview of the processes of the AI platform and machine learning model 150 according to certain embodiments of the invention, which are directed to analyzing an inquiry from the user/person struggling with addiction and determining an appropriate response. Generally, the AI platform and machine learning model 150 decompose the inquiry from the person 101 using multiple stages. The first stage identifies the specific words used in the inquiry and searches for words that have similar meanings. The next stage examines the sequence of words in the inquiry to better understand the meaning and intent behind the inquiry. After the meaning of the inquiry is understood, the AI platform and machine learning model 150 determine a relevant action to be performed in the form of a response to the inquiry. Finally, the AI platform and machine learning model 150 combine all of the previous inquiries and responses to generate a more appropriate and relevant response, which guides the person 101 through the addiction recovery process.

In one embodiment of the invention, the system 100 is configured to provide “real-time” responses, feedback and/or status reports regarding the progress of the person in an addiction recovery program, to any third-parties including, but not limited to, agencies, courts, schools and treatment facilities. The described information (i.e., responses, feedback and/or status reports) can be provided to any designated third-party through any standard communication channels using known wired and wireless data communication techniques.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array), an ASIC (application-specific integrated circuit), a GPU (graphical programming unit), or a CPU (central processing unit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone, running a messaging application, and receiving responsive messages from the user in return.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. In some circumstances, quantum computing/processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A computer-implemented method for providing a digital virtual sponsor to facilitate addiction recovery comprising:
receiving a first inquiry from a person struggling with addiction;
analyzing the first inquiry to determine a response category using a natural language machine learning model;
determining at least one appropriate response to the first inquiry as part of a recovery plan based on a scripted interaction associated with the response category and a machine learning model trained for recovery plan responses;
providing the at least one response to the first inquiry to the person struggling with addiction;
updating a knowledge base containing information to help the person struggling with addiction with information associated with the determined provided at least one response to the first inquiry;
after receiving a second inquiry from the person struggling with addiction, retraining the machine learning model trained for recovery plan responses to determine at least one response to the second inquiry on a basis of the updated knowledge base, and
outputting the determined at least one response to the second inquiry as a part of the recovery plan by using the retrained machine learning model trained for recovery plan responses, wherein
the retrained machine learning model trained for recovery plan responses facilitates the determination of the at least one response to the second inquiry that is personalized to the person struggling with addiction, based on a result of analyzing information directed to a plurality of previous interactions with the person and the knowledge base comprising information on actual experiences of a plurality of other persons with successful recovery from addiction,
the retrained machine learning model trained for recovery plan responses further determines the at least one response to the second inquiry by:
dividing contents of the second inquiry into a plurality of tokens corresponding to a plurality of words;
converting each of the plurality of tokens associated with one of the plurality of words into a plurality of vectors;
comparing the plurality of vectors of the corresponding plurality of tokens with the updated knowledge base to determine an intent of the second inquiry of the person; and
searching for the determined at last one response to the second inquiry based on the determined intent of the second inquiry and the updated knowledge base,
the plurality of previous interactions with the person comprises the received first inquiry and the determined provided at least one response to the first inquiry,
the result of analyzing information used to determine the at least one response to the second inquiry is further based on a determined match of a same attribute between the plurality of previous interactions with the person and the information on actual experiences of the plurality of other persons with successful recovery from addiction, and
the outputted at least one response to the second inquiry is tailored to the person and based on the information on actual experiences of the plurality of other persons with successful recovery from addiction to optimize success of the recovery plan.

2. The computer-implemented method of claim 1, further comprising determining whether to issue a digital reward on a basis of the recovery plan and an action performed by the person in response to the at least one appropriate response to the first inquiry, and issuing the digital reward after a determination that the person follows the recovery plan on a basis of the at least one appropriate response to the first inquiry.

3. The computer-implemented method of claim 1, wherein each of the first and second inquiries is a request for information, a statement for motivation, or a warning sign that the person needs help to continue a recovery process.

4. The computer-implemented method of claim 1, wherein the first inquiry is a spoken request and analyzing the first inquiry includes using natural language processing to determine the response category.

5. The computer-implemented method of claim 1, wherein the scripted interaction is a set of interactive dialogue prompts that is created using a predefined recovery plan and a profile associated with the person.

6. The computer-implemented method of claim 4, wherein the scripted interaction is updated using the machine learning model based on responses from previous interactive dialogue with the person and updates to the person's profile.

7. A system for providing a digital virtual sponsor to facilitate addiction recovery comprising:
a digital assistant platform that allows communication with a person struggling with addiction using natural language conversations;
a knowledge base containing information about resources, activities, and recovery plans to help the person struggling with addiction;
a processor to execute a program; and
a memory to store the program which, when executed by the processor, the processor performs processes of,
receiving a first inquiry from a user using the digital assistant platform;
analyzing the first inquiry to determine a response category using a natural language machine learning model;
determining at least one appropriate response to the first inquiry as part of a recovery plan based on a scripted interaction associated with the response category and a machine learning model trained for recovery plan responses using the knowledge base;
providing the at least one response to the first inquiry to the user using the digital assistant platform;
updating the knowledge base with information associated with the determined provided at least one response to the first inquiry;
after receiving a second inquiry from the user using the digital assistant platform, retraining the machine learning model trained for recovery plan responses to determine at least one response to the second inquiry on a basis of the updated knowledge base, and
outputting the determined at least one response to the second inquiry as a part of the recovery plan by using the retrained machine learning model trained for recovery plan responses, wherein
the retrained machine learning model trained for recovery plan responses facilitates the determination of the at least one response to the second inquiry that is personalized to the user, based on a result of analyzing information directed to a plurality of previous interactions with the user and the knowledge base comprising information on actual experiences of a plurality of other persons with successful recovery from addiction,
the retrained machine learning model trained for recovery plan responses further determines the at least one response to the second inquiry by:
dividing contents of the second inquiry into a plurality of tokens corresponding to a plurality of words;
converting each of the plurality of tokens associated with one of the plurality of words into a plurality of vectors;
comparing the plurality of vectors of the corresponding plurality of tokens with the updated knowledge base to determine an intent of the second inquiry of the person; and
searching for the determined at last one response to the second inquiry based on the determined intent of the second inquiry and the updated knowledge base,
the plurality of previous interactions with the user comprises the received first inquiry and the determined provided at least one response to the first inquiry,
the result of analyzing information used to determine the at least one response to the second inquiry is further based on a determined match of a same attribute between the plurality of previous interactions with the user and the information on actual experiences of the plurality of other persons with successful recovery from addiction, and
the outputted at least one response to the second inquiry is tailored to the user and based on the information on actual experiences of the plurality of other persons with successful recovery from addiction to optimize success of the recovery plan.

8. The system of claim 7, wherein the processor further performs processes of: determining whether to issue a digital reward on a basis of the recovery plan and an action performed by the user in response to the at least one appropriate response to the first inquiry, and issuing the digital reward after a determination that the user follows the recovery plan on a basis of the at least one appropriate response to the first inquiry.
